(19) United States
(12) Patent Application Publication
(10) Pub. No.: US 2011/0027363 A1
(43) Pub. Date: Feb. 3, 2011

(54) Nouvelle forme d'administration de proteines osteogeniques

(75) Inventors: Remi SOULA, Lyon (FR); Olivier SOULA, Meyzieu (FR); Gerard SOULA, Meyzieu (FR)

Correspondence Address: OLIFF & BERRIDGE, PLC, P.O. BOX 320850, ALEXANDRIA, VA, 22320-4850, US

(73) Assignee: ADOCIA, LYON (FR)

(21) Appl. No.: 12/591,447

(22) Filed: Nov. 19, 2009

Related U.S. Application Data
(60) Provisional application No. 61/213,938, filed on Jul. 31, 2009.

(30) Foreign Application Priority Data
Jul. 31, 2009 (FR) 09/03802

(51) Int. Cl. A61K 38/18; A61K 38/02; A61K 38/16

(52) U.S. Cl. 424/484; 514/7.6; 514/8.1; 514/8.2; 514/8.8; 514/17.2

(57) ABSTRACT

Osteogenic compositions are formed from a coprecipitate that contains at least one insoluble calcium salt and at least one osteogenic protein, the coprecipitate being in divided form. A process for preparing the coprecipitate in divided form contains at least one insoluble calcium salt and at least one complex between an osteogenic protein and a polysaccharide. The invention also relates to the formulations, pharmaceutical products, kits and medical devices comprising the coprecipitate.

The present invention relates to the field of osteogenic formulations and more particularly formulations of osteogenic proteins belonging to the family of Bone Morphogenetic Proteins, BMPs.

Bone Morphogenetic Proteins (BMPs) are growth factors involved in osteo-induction mechanisms. BMPs, also known as osteogenic proteins (OPs), were initially characterized by Urist in 1965 (Urist MR. Science 1965; 150, 893). These proteins isolated from cortical bone have the capacity of inducing bone formation in a large number of animals (Urist MR. Science 1965; 150, 893).

BMPs are expressed in the form of propeptides, which, after post-translational maturation, have a length of between 104 and 139 residues. There is large sequence homology between them and they have similar three-dimensional structures. In particular, they contain six cysteine residues involved in intramolecular disulfide bridges forming a “cysteine knot” (Scheufler C. 2004 J. Mol. Biol. 1999; 287, 103; Schlunegger M P, J. Mol. Biol. 1993; 231, 445). Some of them contain a seventh cysteine also involved in an intermolecular disulfide bridge, which is the origin of the formation of the dimer (Scheufler C. 2004 J. Mol. Biol. 1999; 287:103.).

In their active form, BMPs assemble into homodimers, or even into heterodimers, as has been described by Israel et al. (Israel DI, Growth Factors. 1996; 13(3-4), 291). Dimeric BMPs interact with transmembrane receptors of BMPR type (Mundy et al. Growth Factors, 2004, 22 (4), 233). This recognition is the origin of an intracellular signal cascade especially involving the Smad proteins, thus resulting in activation or repression of the target genes.

With the exception of BMP 1 and 3, BMPs play a direct and indirect role in the differentiation of mesenchymal cells, causing their differentiation into osteoblasts (Cheng H., J. Bone and Joint Surgery, 2003, 85A 1544-1552). They also have chemotactic properties and induce proliferation, differentiation and angiogenesis.

Certain recombinant human BMPs, and especially rhBMP-2 and rhBMP-7, have clearly shown a capacity to induce bone formation in vivo in man and have been approved for certain medical applications. Thus, recombinant human BMP-2, dibotermine alpha according to the international nonproprietory name, is formulated in products marketed under the name InFUSE® in the United States and InductOs® in Europe. This product is prescribed in the fusion of lumbar vertebrae and tibial bone regeneration for “non-union” fractures. In the case of InFUSE® for the fusion of lumbar vertebrae, the surgical intervention consists firstly in soaking a collagen sponge with a solution of rhBMP-2, and then placing the sponge in a hollow cage, LT cage, implanted beforehand between the vertebrae.

Recombinant human BMP-7, eptotermine alpha according to the international nonproprietory name; has the same therapeutic indications as BMP-2 and is the basis of two products: OP-1 Implant for open fractures of the tibia, and OP-1 Putty for the fusion of lumbar vertebrae. OP-1 Implant is composed of a powder containing rhBMP-7 and collagen to be taken up in a 0.9% saline solution. The paste obtained is then applied to the fracture during a surgical intervention. OP-1 Putty is in the form of two powders: one containing rhBMP-7 and collagen, the other carboxymethylcellulose (CMC). During a surgical intervention, the CMC is reconstituted with a 0.9% saline solution and mixed with the rhBMP-7 and the collagen. The paste thus obtained is applied to the site to be treated.

The administration of osteogenic proteins is a major problem on account of their instability and of the need that arises to obtain osteogenic formulations containing a minimal amount of osteogenic protein, so as to avoid the side effects generated by high concentrations of these proteins, and also on account of the cost of these proteins.

Many formulations have been and are being developed, for instance those cited in the review by Seeherman (Seeherman, H. et al., Spine 2002, 27 (16 Suppl. 1), S16-S23.), in which the importance of the nature of the delivery system is emphasized.

The delivery systems used must make it possible to increase the retention time of the proteins at the site of administration, to obtain total release of the amount of protein used and to avoid an overly abrupt release that may lead to diffusion outside the site of administration.

The delivery system used must also be able to serve as a matrix for bone growth at the site to be treated, while at the same time defining the limits of this bone growth at the site to be treated.

Four types of material are used in delivery systems at the present time: natural polymers, synthetic polymers, inorganic materials, and mixtures of these materials.

However, none of the systems developed has made it possible to significantly reduce the dose of BMP. This is associated, inter alia, either with the instability of the protein in the formulation, or with its poor bioavailability on account of the structure of the support.

As regards natural polymers, collagen, hyaluronans, fibrin, chitosans, alginates and other natural polysaccharides are used.

Although recombinant collagen-based sponges make it possible to overcome most of the known drawbacks of this natural polymer, the introduction of osteogenic protein into the sponges is not satisfactory at the present time.

The other natural polysaccharides in the form of hydrogels essentially have the defect of being resorbed too quickly, unless they are crosslinked beforehand in the form of gels, which leads to the same drawbacks as those mentioned previously for the collagen sponges.

As regards synthetic polymers, the ones most commonly used are poly(α-hydroxy acid) polymers such as polylactide (PLA), polyglycolide (PLG) and copolymers thereof (PLGA).

The major drawbacks of these polymers are the lowering of the pH due to their degradation and the inflammation reactions they may induce.

As regards inorganic materials, delivery systems combining calcium phosphates with a osteo-inducing protein have been developed.

Among these, mention will be made of calcium phosphate-based ceramics, such as hydroxyapatite (HAP) and tricalcium phosphate (TCP), and “non-ceramic” calcium phosphates, for instance calcium phosphate-based cements (CPCs).

It has been known since the 1970s that calcium phosphate ceramics may be of value in bone reconstruction, as is recalled in the review by M. Bohner (Bohner, M., Injury 2000, 31 Suppl. 4, 37-47.).

However, it is accepted that the effective dose of BMP-2 is higher in a ceramic than in a collagen sponge. A clinical study of posterolateral fusion in man (Boden, S. D. et al., Spine 2002, 27 (23), 2662-2673.) reports that the dose of BMP-2 (40 mg) is higher with BCP granules (60% HAP and 40% TCP), a product developed by the company Medtronic Sofamor, than in a collagen sponge not containing calcium phosphate (12 mg).

In order to overcome this drawback, a very large number of systems have been developed based on non-ceramic calcium phosphate, among which are calcium phosphate cements. Cements were discovered in the 1980s by Brown and Chow and correspond to the following definition: “Calcium phosphate cements are formed from an aqueous solution and from one or more calcium phosphates. When mixed together, the calcium phosphate(s) dissolve(s) and precipitate(s) as a less soluble calcium phosphate salt. During the precipitation, the calcium phosphate crystals enlarge and overlap, which leads to the mechanical rigidity of the cement.” (Bohner, M., Injury 2000, 31 Suppl. 4, 37-47.).

An article by Kim (Kim, H. D. et al., Methods Mol Biol. 2004, 238, 49-64.) describes the use of a cement developed by the company Etex, alpha-BSM, with BMP-2. This novel product does indeed lead to bone-inducing activity of the matrix.

However, the BMP-2 introduced into this matrix loses a large part of its activity, leading to the need to increase the amount of BMP-2 incorporated. Thus, a dose of 40 μg of BMP-2 is employed in the model of formation of ectopic bone in rats, instead of the 20 μg of BMP-2 employed in a collagen sponge.

In point of fact, cements have two drawbacks. Firstly, the calcium phosphate(s) that are their precursors must be synthesized beforehand under conditions that are incompatible with proteins. Thus, U.S. Pat. No. 5,650,176 describes the reaction conditions necessary for the preparation of amorphous calcium phosphate, which is one of the compounds of alpha-BSM. These conditions are incompatible with proteins since a very large amount of sodium hydroxide is used. Furthermore, these products require vigorous purification since toxic compounds such as calcium nitrate are used.

Other examples of cements such as those described by the company Graftys in patent EP 1 891 984 A1 are obtained under conditions that are incompatible with proteins since dichloromethane is used in the synthesis of the calcium phosphate. The cements described by the company Lisopharm in patent US 2009/0 155 320 are obtained in the presence of calcium hydroxide, which is also incompatible with proteins.

Furthermore, in general, the formation of cement is obtained by reacting a soluble calcium phosphate salt with a solid calcium phosphate salt treated at more than 400° C. in order to make it reactive. The reaction between these two compounds is uncontrolled, mainly exothermic, and leads to a cement of monolithic structure that sequesters protein into its bulk.

In U.S. Pat. No. 563,461, mention is made of the presence of “reactive holes” in the solid, without stating whether this is harmful to the chemical stability of BMP-2.

In order to reduce the losses of protein in the mass of solid formed, it has been described in U.S. Pat. No. 5,650,176 that it is advantageous to add to the reaction mixture effervescent compounds capable of reducing the “monolithic” nature of the cement.

Despite these improvements, the observation cannot be avoided that the amounts of protein required to obtain a bone formation in the model of ectopic rat remain high.

As regards mixed systems, they have not to date made it possible to overcome the problems mentioned above.

In summary, the systems described in the prior art concerning the use of synthetic polymers, natural polymers or inorganic materials such as calcium phosphate cements or ceramics do not fully satisfy the specifications imposed for applications in bone repair.

The Applicant has, to its credit, developed a novel approach that consists in placing osteogenic protein in contact with soluble calcium salts and soluble phosphate salts, which can satisfy the specifications imposed for applications in bone repair.

This novel approach makes it possible, on the one hand, to precipitate the protein, while avoiding any chemical degradation on contact with the reagents present, and, on the other hand, to coprecipitate it with an insoluble calcium salt, preferably calcium phosphate, said coprecipitate being in divided form, which very markedly limits the losses in the mass of solid as observed with cements.

Thus, the Applicant has developed novel osteogenic compositions composed of a coprecipitate that contains at least one insoluble calcium salt and at least one osteogenic protein, said coprecipitate being in divided form.

The conjunction of these two events makes it possible to obtain very osteogenic formulations containing much smaller amounts of protein.

These novel compositions thus have the advantage of containing smaller amounts of protein, which is the major objective, in order to reduce the side effects after administration to patients.

Furthermore, they allow a reduction in the costs of treatments by reducing the amount of protein, since these proteins are very expensive.

Provisional patent applications 61/129,023 and 61/129,617 in the name of the Applicant are known, the entire contents of which applications are incorporated into the present patent application by reference, which describe and claim osteogenic compositions comprising at least one osteogenic protein, a soluble salt of a divalent cation and a matrix.

Provisional patent applications 61/129,011 and 61/129,618 in the name of the Applicant are known, the entire contents of which applications are incorporated into the present patent application by reference, which describe and claim osteogenic compositions comprising at least one osteogenic protein, at least one angiogenic protein, a soluble salt of a divalent cation, optionally an anionic polysaccharide and optionally a matrix.

Provisional patent application U.S. 61/193,217 filed on Nov. 6, 2008 in the name of the Applicant is known, the entire content of which is incorporated into the present patent application by reference, which describes and claims osteogenic compositions comprising at least one osteogenic protein, a soluble salt of an at least divalent cation, and a polymer forming a hydrogel.

As regards the present invention, the Applicant has also developed the process for preparing the coprecipitate, in divided form, containing at least one insoluble calcium salt and at least one osteogenic protein.

The invention also relates to the formulations, the pharmaceutical products and the medical devices comprising said coprecipitate.

The compositions and kits for using this process and for obtaining the coprecipitate are also inventions described hereinbelow.

The coprecipitation is obtained by:

precipitation of the osteogenic protein by addition of a solution of a salt of calcium ions,

precipitation of the calcium ions by addition of a composition comprising at least one soluble salt of an anion capable of forming an insoluble calcium salt at a given pH.

In one embodiment, the precipitation of the calcium salt takes place in the form of calcium phosphate, by addition of a soluble phosphate solution.

The nature and form of the coprecipitate may vary as a function of the pH of the solutions placed in contact, since calcium phosphate salts have different solid phases as a function of the pH.

The invention relates to a coprecipitate consisting of at least one osteogenic protein in its undissolved form and at least one insoluble calcium salt, said coprecipitate being in divided form.

In one embodiment, the insoluble calcium salt is chosen from the group consisting of calcium orthophosphates in anhydrous or hydrated form, alone or as a mixture.

In one embodiment, the coprecipitate also comprises at least one insoluble calcium salt chosen from the group consisting of calcium oxalate, calcium ascorbate, calcium carbonate and calcium sulfate.

Said insoluble calcium salts may be mixed salts formed between cationic calcium ions and anionic ions such as mono-, di- or tribasic phosphates, polysaccharide carboxylates, carbonates, hydroxides and the possible anions borne by bases.

Calcium orthophosphates are salts that result from the neutralization of the various acidities of phosphoric acid with calcium salts, and, according to the literature, the pKa values range from 2.12 to 12.67 at 25° C.

The main insoluble calcium orthophosphates are dicalcium phosphates, DCP, anhydrous or dihydrated, octacalcium phosphates, OCP, tricalcium phosphates, TCP, phosphocalcic hydroxyapatites, HAP or PCA, and tetracalcium phosphate, TTCP.

This coprecipitation as a function of the desired effect is optionally obtained in the presence of a base that allows the pH to be adjusted to a predetermined value.

This coprecipitation makes it possible to obtain a solid chemical composition, in divided form, which especially makes it possible to control the delivery of the osteogenic protein contained in the composition.

This solid chemical composition, in divided form, is obtained spontaneously under room temperature conditions, and its divided state is stable under physiological conditions.

In one embodiment, the coprecipitate results from simultaneous precipitations.

In one embodiment, the coprecipitate results from sequential precipitations.

In one embodiment, the invention consists of a kit for preparing an osteogenic implant comprising at least:
a—a composition comprising at least one osteogenic protein,
b—a composition comprising at least one soluble calcium salt,
c—a composition comprising at least one soluble salt of an anion capable of forming an insoluble calcium salt.

In one embodiment, the kit also comprises an additional composition comprising at least one base.

In one embodiment, a second base may be added to compositions b or c.

Some of these compositions may be combined before the formation of the coprecipitate in order to reduce the number of vials.

The composition comprising the osteogenic protein may also comprise the soluble salt of an anion capable of forming an insoluble calcium salt and/or a base.

In one embodiment, the composition comprising the soluble calcium salt may also comprise a base.

In one embodiment, the kit comprises:
a—a composition comprising at least one osteogenic protein,
b—a composition comprising at least one soluble salt of an anion capable of forming an insoluble calcium salt,
c—a composition comprising at least one soluble calcium salt,
d—a composition comprising at least one base.

In this embodiment, a second base, which may be identical to or different than the base of composition d, may be added to compositions b and c.

In one embodiment, the kit comprises
a—a composition comprising at least one osteogenic protein,
b—a composition comprising at least one soluble calcium salt and at least one base,
c—a composition comprising at least one soluble salt of an anion capable of forming an insoluble calcium salt.

According to this embodiment, a second base, which may be identical to or different than the base of composition b, may be added to composition c.

In one embodiment, the kit comprises
a—a composition comprising at least one osteogenic protein and at least one soluble salt of an anion capable of forming an insoluble calcium salt,
b—a composition comprising at least one soluble calcium salt,
c—a composition comprising at least one base.

In this embodiment, a second base, which may be identical to or different than the base of composition c, may be added to composition b.

In one embodiment, the kit comprises
a—a composition comprising at least one osteogenic protein and at least one soluble salt of an anion capable of forming an insoluble calcium salt,
b—a composition comprising at least one soluble calcium salt and at least one base.

In this embodiment, a second base, which may be identical to or different than the base of composition b, may be added to composition a.

In one embodiment, the kit comprises
a—a composition comprising at least one osteogenic protein,
b—a composition comprising at least one soluble calcium salt,
c—a composition comprising at least one soluble salt of an anion capable of forming an insoluble calcium salt and at least one base.

In one embodiment, the kit comprises
a—a composition comprising at least one osteogenic protein, at least one soluble salt of an anion capable of forming an insoluble calcium salt and at least one base,
b—a composition comprising at least one soluble calcium salt.

In this embodiment, a second base, which may be identical to or different than the base of composition a, may be added to composition b.

In one embodiment, the composition comprising at least one osteogenic protein also comprises at least one growth factor with chemo-attracting and angiogenic power.

In one embodiment, the kit also comprises at least one organic matrix or a mineral matrix or a mixed matrix.

In one embodiment, the compositions constituting the kit are aqueous solutions.

In one embodiment, the compositions constituting the kit are lyophilizates.

In one embodiment, some of the compositions constituting the kit are lyophilizates.

In this embodiment, the lyophilizates are rehydrated before being reacted, with water or one of the other compositions in solution.

Thus, for example, the composition comprising the osteogenic protein in lyophilizate form may be rehydrated with the solution comprising a soluble salt of an anion capable of forming an insoluble calcium salt and/or a base.

In one embodiment, the pharmaceutical formulations and products comprising said coprecipitate are aqueous suspensions.

In one embodiment, the pharmaceutical formulations and products comprising said coprecipitate are lyophilizates.

In this embodiment, the lyophilizates are rehydrated before use, with physiological saline or blood.

The term “osteogenic protein” means an osteogenic growth factor or BMP, alone or in combination, the BMP being chosen from the group of therapeutically active BMPs (Bone Morphogenetic Proteins).

More particularly, the osteogenic proteins are chosen from the group consisting of BMP-2 (dibotermine-alpha), BMP-4, BMP-7 (eptotermine-alpha), BMP-14 and GDF-5, alone or in combination.

The BMPs used are recombinant human BMPs, obtained according to the techniques known to those skilled in the art or purchased from suppliers, for instance the company Research Diagnostic Inc. (USA).

The term “growth factor with chemo-attracting and angiogenic power” means proteins such as PDGF, especially PDGF-BB, VEGF or FGF, especially FGF-2.

In one embodiment, the osteogenic protein is chosen from the group consisting of BMP-2 (dibotermine-alpha), BMP-4, BMP-7 (eptotermine-alpha), BMP-14 and GDF-5, alone or in combination, and the at least one growth factor with chemo-attracting and angiogenic power is PDGF.

In one embodiment, the composition comprises at least BMP-2 and PDGF-BB.

In one embodiment, the composition comprises at least BMP-7 and PDGF-BB.

In one embodiment, the composition comprises at least GDF-5 and PDGF-BB.

In one embodiment, the osteogenic protein is chosen from the group consisting of BMP-2 (dibotermine-alpha), BMP-4, BMP-7 (eptotermine-alpha), BMP-14 and GDF-5, alone or in combination, and the at least one growth factor with chemo-attracting and angiogenic power is VEGF.

In one embodiment, the osteogenic protein is chosen from the group consisting of BMP-2 (dibotermine-alpha), BMP-4, BMP-7 (eptotermine-alpha), BMP-14 and GDF-5, alone or in combination, and the at least one growth factor with chemo-attracting and angiogenic power is FGF.

The soluble calcium salt is a calcium salt whose anion is chosen from the group consisting of chloride, D-gluconate, formate, D-saccharate, acetate, L-lactate, glutamate and aspartate.

In one embodiment, the soluble calcium salt is calcium chloride.

The term “soluble salt of an anion capable of forming a precipitate with the calcium ion” means a soluble salt whose anion is chosen from the group consisting of phosphate anions comprising the phosphate ion PO43-, the hydrogen phosphate ion HPO42- and the dihydrogen phosphate ion H2PO4-.

In one embodiment, a second anion chosen from the group consisting of oxalate, ascorbate, carbonate and sulfate anions is also added to the composition comprising a phosphate anion.

The soluble salts of an anion that can form a precipitate with the calcium ion are chosen from the group consisting of sodium phosphates, sodium oxalate, sodium ascorbate, sodium carbonate, sodium sulfate and sodium hydrogen carbonate.

The bone deficit determines the volume of the solutions that can be used; there is thus a need to reduce the volumes of each solution and thus the concentrations of the constituents below their solubility limit.

Furthermore, the solutions must be stored at about 4° C. for stability reasons, which further lowers the solubility limit.

In order to neutralize the acidic compounds present in the mixture, the bases are chosen from mineral and organic bases.

Among the mineral bases, mention will be made of sodium hydroxide, sodium hydrogen carbonate and sodium carbonate.

Among the organic bases, mention will be made of amines and deprotonated amino acids.

Among the organic bases, mention will be made of imidazole and derivatives thereof, especially histidine, proline, ethanolamine and serine.

In one embodiment, an organic matrix may be used in order to promote repair; it is chosen from matrices based on purified, sterilized and crosslinked natural collagen.

Natural polymers such as collagen are components of the extracellular matrix that promote cell attachment, migration and differentiation. They have the advantage of being extremely biocompatible and are degraded by enzymatic digestion mechanisms. Collagen-based matrices are obtained from fibrillar collagen of type I or IV extracted from bovine or porcine tendon or bone. These collagens are first purified, before being crosslinked and then sterilized.

They may also be obtained by resorption in acidic medium of autologous bone, leading to the loss of most of the mineralized components, but to preservation of the collagen or non-collagen proteins, including growth factors. These demineralized matrices may also be prepared in inactive form after extraction with chaotropic agents. These matrices are essentially composed of insoluble and crosslinked collagen of type I.

Mixed materials may also be used, for example a matrix that combines collagen and inorganic particles. These materials may be in the form of a composite material with reinforced mechanical properties or in the form of a “putty” in which the collagen acts as a binder.

The inorganic materials that may be used essentially comprise calcium phosphate-based ceramics such as hydroxyapatite (HA), tricalcium phosphate (TCP), biphasic calcium phosphate (BCP) or amorphous calcium phosphate (ACP), the main value of which is that they have a chemical composition very similar to that of bone. These materials have good mechanical properties and are immunologically inert. These materials may be in various forms, such as powders, granulates or blocks. These materials have very different degradation rates as a function of their composition. Thus, hydroxyapatite degrades very slowly (several months), whereas tricalcium phosphate degrades more quickly (several weeks). It is for this reason that biphasic calcium phosphates were developed, since they have intermediate resorption rates. These inorganic materials are known to be mainly osteo-conducting.

In one embodiment, the organic matrix is a crosslinked hydrogel.

A crosslinked hydrogel is obtained by crosslinking polymer chains. The inter-chain covalent bonds define an organic matrix. The polymers that may be used to constitute an organic matrix are described in the review by Hoffman entitled Hydrogels for biomedical applications (Adv. Drug Deliv. Rev, 2002, 43, 3-12).

In one embodiment, the implant may comprise a non-crosslinked hydrogel.

The term “non-crosslinked hydrogel” means a hydrophilic three-dimensional polymer network capable of adsorbing a large amount of water or of biological liquids (Peppas et al., Eur. J. Pharm. Biopharm. 2000, 50, 27-46). This hydrogel is formed by physical interactions and is therefore not obtained by chemical crosslinking of the polymer chains.

The list of polymers forming hydrogels is very long, and a large but not exhaustive list is given in the review by Hoffman entitled Hydrogels for biomedical applications (Adv. Drug Deliv. Rev., 2002, 43, 3-12). Among these polymers are synthetic polymers and natural polymers. Another review covering polysaccharides that form hydrogels makes it possible to choose a polymer that is useful for the invention (Alhaique et al. J. Control. Release, 2007, 119, 5-24).

In one embodiment, the polymer forming a hydrogel is chosen from the group of synthetic polymers, including copolymers of ethylene glycol and of lactic acid, copolymers of ethylene glycol and of glycolic acid, poly(N-vinylpyrrolidone), polyvinylic acids, polyacrylamides and polyacrylic acids.

In one embodiment, the polymer forming a hydrogel is chosen from the group of natural polymers, including hyaluronic acid, keratan, pullulan, pectin, dextran, cellulose and cellulose derivatives, alginic acid, xanthan, carrageenan, chitosan, chondroitin, collagen, gelatin, polylysine, fibrin and biologically acceptable salts thereof.

In one embodiment, the natural polymer is chosen from the group of polysaccharides forming hydrogels, including hyaluronic acid, alginic acid, dextran, pectin, cellulose and derivatives thereof, pullulan, xanthan, carrageenan, chitosan, chondroitin, and biologically acceptable salts thereof.

In one embodiment, the natural polymer is chosen from the group of polysaccharides forming hydrogels, including hyaluronic acid, alginic acid, and biologically acceptable salts thereof.

In one embodiment, the hydrogel may be prepared just before implanting.

In one embodiment, the hydrogel may be prepared and stored in a prefilled syringe in order then to be implanted.

In one embodiment, the hydrogel may be prepared by rehydration of a lyophilizate just before implanting or may be implanted in dehydrated form

Among the various matrices which can be used, mention will be made, for example, of collagen sponges such as Helistat® (Integra LifeSciences, Plainsboro, N.J.), DBMs (Demineralized Bone Matrix) alone or as a mixture with other organic materials such as polysaccharides, glycerol or gelatins such as Osteofil® (Medtronic), Allomatrix® (Wright), Grafton® (Osteotech), DBX® (MTF/Synthes), Bioset® (Regeneration Technologies), matrices consisting of mineral phases such as Vitoss® (Orthivista), Osteoset® (Wright) or mixed matrices such as Healos® (DePuy Orthopaedics), MasterGraft® (Medtronic), CopiOs® (Zimmer), Sunnmax Collagen Bone Graft Matrix (Sunmax).

The system after formation of the coprecipitate is formed from two phases, a liquid phase and a solid phase.

In the rest of the specification, when the notion of volume is employed, it is the total volume comprising the two phases.

The amounts per unit volume in the product resulting after mixing together the compositions of the various forms of the kit are given below.

In one embodiment, the total amounts of the various proteins per unit volume are between 0.01 mg and 2 mg, preferably between 0.05 mg and 1.5 mg and more preferably between 0.2 mg and 1.5 mg per ml of suspension obtained.

The total amounts of phosphates per unit volume are between 0.02 mmol and 0.5 mmol and preferably between 0.05 and 0.25 mmol per ml of suspension obtained.

The total amounts of calcium per unit volume are between 0.1 mmol and 1 mmol, preferably between 0.05 and 1 mmol and more preferably between 0.1 mmol and 0.5 mmol per unit volume.

The percentage of calcium ions in the solid phase is between 60% and 95% of the calcium ions introduced.

The amounts of base used correspond to about 0.1 to 2 equivalents relative to the protons provided by the phosphate ions.

As a function of volumes used and of the number of compositions, the amounts used in the starting compositions may be determined by calculation. This may be performed for the various embodiments of the kits.

In one embodiment, for a vertebral implant, the doses of osteogenic growth factor will be between 0.01 mg and 20 mg, preferably between 0.05 mg and 8 mg, preferably between 0.1 mg and 4 mg and more preferably between 0.1 mg and 2 mg, whereas the doses commonly accepted in the literature are between 8 and 12 mg.

In one embodiment, for a vertebral implant, the doses of angiogenic growth factor will be between 0.05 mg and 8 mg, preferably between 0.1 mg and 4 mg and more preferably between 0.1 mg and 2 mg.

In one embodiment, for the formulation of an implant comprising the coprecipitate according to the invention, a kit comprising three vials is prepared, said vials containing:
in the first, between 2 and 10 mg of osteogenic protein in lyophilized form,
in the second, between 2 and 6 ml of a solution of an equimolar mixture of sodium hydrogen phosphate Na2HPO4 and of sodium dihydrogen phosphate NaH2PO4 with a concentration of between 0.15 and 0.50 M,
in the third, between 2 and 6 ml of a calcium chloride solution at a concentration of between 0.25 and 0.90 M.

In one embodiment, the second vial also contains a sodium bicarbonate solution at a concentration of between 0.20 and 0.8 M.

In one embodiment, the second and third vials also contain a histidine solution at a concentration of between 0.02 and 0.2 M.

In one embodiment, the third vial also contains a proline solution at a concentration of between 0.05 and 0.3 M.

The solutions are added simultaneously or successively before implanting, to a collagen sponge with a volume of between 15 and 30 ml.

In one embodiment, for the formulation of an implant comprising the coprecipitate according to the invention, three solutions are mixed together, comprising:
in the first, a volume of between 1 and 3 ml containing an osteogenic protein at a concentration of between 0.33 and 2 mg/ml,
in the second, a volume of between 1 and 3 ml of an equimolar mixture of sodium hydrogen phosphate Na2HPO4 and of sodium dihydrogen phosphate NaH2PO4 with a concentration of between 0.05 and 0.15 M,
in the third, a volume of between 1 and 3 ml containing calcium chloride at a concentration of between 0.25 and 0.50 M.

In one embodiment, a sodium bicarbonate solution at a concentration of between 0.20 and 0.4 M is added to the mixture obtained.

In one embodiment, a histidine solution at a concentration of between 0.02 and 0.15 M is added to the mixture obtained.

In one embodiment, a proline solution at a concentration of between 0.05 and 0.3 M is added to the mixture obtained.

The mixture comprising the coprecipitate according to the invention is then lyophilized.

At the time of use, it is rehydrated with injectable water and/or blood to about 35% of the initial volume.

The invention also relates to the use of the compositions of the invention by implantation, for example, for filling bone defects, for performing vertebral fusions or maxillo-facial repairs, or for treating bone fractures, in particular of the pseudarthrosis type.

The invention also relates to the use of the compositions according to the invention as bone implants.

In one embodiment, the compositions may be used in combination with a prosthetic device of the vertebral prosthesis or vertebral fusion cage type.

The invention also relates to therapeutic and surgical methods using the compositions in bone reconstruction.

In these various therapeutic uses, the size of the matrix and the amount of osteogenic growth factor depend on the volume of the site to be filled.

Examples of kits are given as non-limiting guides.

### EXAMPLE 1

### Preparation of a Kit Containing 4 Vials

Kit 1: A kit of 4 vials comprises a vial containing osteogenic protein in lyophilized or solution form, a vial containing a soluble calcium salt in lyophilized or solution form, a vial containing a soluble phosphate salt in lyophilized or solution form and a vial containing a base in lyophilized or solution form.

### EXAMPLE 2

### Preparation of a Kit Containing 3 Vials

Kit 2: A kit of 3 vials comprises a vial containing osteogenic protein in lyophilized or solution form, a vial containing a soluble calcium salt in lyophilized or solution form and a vial containing a soluble phosphate salt in lyophilized or solution form.

### EXAMPLE 3

### Preparation of a Kit Containing 3 Vials

Kit 3: A kit of 3 vials comprises a vial containing osteogenic protein in lyophilized or solution form, a vial containing a soluble calcium salt and a base in lyophilized or solution form and a vial containing a soluble phosphate salt in lyophilized or solution form.

### EXAMPLE 4

### Preparation of a Kit Containing 3 Vials

Kit 4: A kit of 3 vials comprises a vial containing osteogenic protein in lyophilized or solution form, a vial containing a soluble calcium salt in lyophilized or solution form and a vial containing a soluble phosphate salt and a base in lyophilized or solution form.

### EXAMPLE 5

### Preparation of a Kit Containing 2 Vials

Kit 5: A kit of 2 vials comprises a vial containing osteogenic protein and a soluble phosphate salt in lyophilized or solution form, and a vial containing a soluble calcium salt in lyophilized or solution form.

### EXAMPLE 6

### Preparation of a Kit Containing 2 Vials

Kit 6: A kit of 2 vials comprises a vial containing osteogenic protein, a soluble phosphate salt and a base in lyophilized or solution form, and a vial containing a soluble calcium salt in lyophilized or solution form.

### EXAMPLE 7

### Preparation of a Kit Containing 2 Vials

Kit 7: A kit of 2 vials comprises a vial containing osteogenic protein and a soluble phosphate salt in lyophilized or solution form and a vial containing a soluble calcium salt and a base in lyophilized or solution form.

Examples of solutions or lyophilizates of osteogenic proteins are given as non-limiting guides.

### EXAMPLE 8

### Solution of rhBMP-2 in 1 mM HCl Buffer

10 mL of a 0.15 mg/ml solution of rhBMP-2 are prepared by adding 10 mL of a 1 mM HCl solution to 1.5 mg of lyophilized rhBMP-2 (R&D system). This solution is incubated for two hours at 4° C. and filtered aseptically on a 0.22 μm membrane.

### EXAMPLE 9

### Solution of rhBMP-7 in a 10 mM HCl Buffer

A solution of rhBMP-7 at 3.8 mg/ml is prepared by adding 1 mL of a 1 mM HCl solution to 3.8 mg of lyophilized rhBMP-7. The pH of this solution is 2.2. This solution is incubated for 15 minutes at room temperature and is filtered aseptically on a 0.22 μm membrane.

### EXAMPLE 10

### Solution of rhBMP-7 in a pH 3.5 5% Lactose Buffer

A solution of rhBMP-7 at 3.8 mg/ml is prepared by adding 7.8 mL of a 5% lactose solution whose pH has been set at 3.5 by adding 1 M HCl to 30.3 mg of lyophilized rhBMP-7. The pH of this solution is 3.5. This solution is incubated for 15 minutes at room temperature and filtered aseptically on a 0.22 μm membrane.

### EXAMPLE 11

### Solution of rhGDF-5 in a 10 mM HCl Buffer

Solution 1: 1 mL of an rhGDF-5 solution at 1.5 mg/ml is prepared by adding 1 mL of a 10 mM HCl solution to 1.5 mg of lyophilized rhGDF-5. This solution is incubated for two hours at 4° C. and filtered aseptically on a 0.22 μm membrane.

Examples of preparation of solutions of soluble phosphate salts are given as non-limiting guides.

### EXAMPLE 12

### Sodium Phosphate Solution

Solution 2: A 1 M sodium phosphate solution is prepared in a graduated flask from an equimolar mixture of anhydrous sodium hydrogen phosphate and sodium dihydrogen phosphate (Sigma). This solution is incubated for 30 minutes at room temperature and filtered aseptically on a 0.22 μm membrane.

More dilute sodium phosphate solutions are prepared from the stock solution described above.

Examples of preparation of solutions of soluble calcium salts are given as non-limiting guides.

### EXAMPLE 13

### 2 M Calcium Chloride Solution

Solution 3: A 2 M calcium chloride solution is prepared in a graduated flask from anhydrous or dihydrated calcium chloride (Sigma). This solution is incubated for 30 minutes at room temperature and filtered aseptically on a 0.22 μm membrane.

### EXAMPLE 14

### 0.75 M Calcium Chloride Solution

Solution 4: A 0.75 M calcium chloride solution is prepared by diluting the 2 M calcium chloride solution described in the preceding example. This solution is incubated for 30 minutes at room temperature and filtered aseptically on a 0.22 μm membrane.

### EXAMPLE 15

### 0.75 M Calcium Acetate Solution

Solution 5: A 0.75 M calcium acetate solution is prepared in a graduated flask from calcium acetate (Sigma). This solution is incubated for 30 minutes at room temperature and filtered aseptically on a 0.22 μm membrane.

### EXAMPLE 16

### 0.75 M Calcium Gluconate Solution

Solution 6: A 0.75 M calcium gluconate solution is prepared in a graduated flask from calcium gluconate (Sigma). This solution is incubated for 30 minutes at room temperature and filtered aseptically on a 0.22 μm membrane.

Examples of preparation of solutions of bases are given as non-limiting illustrations.

### EXAMPLE 17

### 1 M Histidine Solution

Solution 7: A 1 M histidine solution is prepared in a 1 L graduated flask by dissolving 155.2 g of L-histidine (Sigma) in the volume of deionized water necessary to reach the graduation mark. This solution is incubated for 30 minutes at room temperature and filtered aseptically on a 0.22 μm membrane.

### EXAMPLE 18

### 2 M Proline Solution

Solution 8: A 2 M proline solution is prepared in a 1 L graduated flask by adding 230.2 g of L-proline (Sigma), 200 mL of 10 N sodium hydroxide and the volume of deionized water necessary to reach the graduation mark. This solution is incubated for 30 minutes at room temperature and filtered aseptically on a 0.22 μm membrane.

### EXAMPLE 19

### 2 M Serine Solution

Solution 9: A 2 M serine solution is prepared in a 1 L graduated flask by adding 210.2 g of L-serine (Sigma), 200 mL of 10 N sodium hydroxide and the volume of deionized water necessary to reach the graduation mark. This solution is incubated for 30 minutes at room temperature and filtered aseptically on a 0.22 μm membrane.

### EXAMPLE 20

### 2 M Glycine Solution

Solution 10: A 2 M glycine solution is prepared in a 1 L graduated flask by adding 150.1 g of L-glycine (Sigma), 200 mL of 10 N sodium hydroxide and the volume of deionized water necessary to reach the graduation mark. This solution is incubated for 30 minutes at room temperature and filtered aseptically on a 0.22 μm membrane.

### EXAMPLE 21

### 2 M Alanine Solution

Solution 11: A 2 M alanine solution is prepared in a 1 L graduated flask by adding 178.2 g of L-alanine (Sigma), 200 mL of 10 N sodium hydroxide and the volume of deionized water necessary to reach the graduation mark. This solution is incubated for 30 minutes at room temperature and filtered aseptically on a 0.22 μm membrane.

### EXAMPLE 22

### 2 M Lysine Solution

Solution 12: A 2 M lysine solution is prepared in a 1 L graduated flask by adding 292.4 g of L-lysine (Sigma), 200 mL of 10 N sodium hydroxide and the volume of deionized water necessary to reach the graduation mark. This solution is incubated for 30 minutes at room temperature and filtered aseptically on a 0.22 μm membrane.

Solutions of lower concentration of these various bases are obtained by dilution either with water or with a solution of the calcium salts described previously.

### EXAMPLE 23

### Sodium Hydrogen Carbonate Solution

A 1.2 M sodium hydrogen carbonate solution is prepared in a graduated flask from anhydrous sodium hydrogen carbonate (Sigma). This solution is incubated for 30 minutes at room temperature and filtered aseptically on a 0.22 μm membrane.

More dilute sodium hydrogen carbonate solutions are prepared from the stock solution described above.

### EXAMPLE 24

### TRIS Solution

A 0.5 M solution of tris(hydroxymethyl)aminomethane is prepared in a graduated flask from ultrapure tris(hydroxymethyl)aminomethane (Sigma) and adjusted to pH 7.4 using 1 M hydrochloric acid. This solution is incubated for 30 minutes at room temperature and filtered aseptically on a 0.22 μm membrane.

Examples of preparation of solutions of osteogenic proteins and of phosphate are given as non-limiting guides.

### EXAMPLE 25

### Preparation of a Solution of rhBMP-2 in the Presence of Sodium Phosphate

Solution 13: A lyophilizate containing 0.77 mg of rhBMP-2 is taken up in 3.86 mL of a 0.45 M sodium phosphate solution obtained by diluting the solution described in Example 12. The concentration of BMP-2 in the solution is 0.2 mg/mL. The solution is incubated for two hours at 4° C. The solution obtained is clear, and is filtered aseptically on a 0.22 μm membrane.

### EXAMPLE 26

### Preparation of a Solution of rhBMP-2 in the Presence of Sodium Phosphate and Sodium Hydrogen Carbonate

Solution 14: A lyophilizate containing 1.0 mg of rhBMP-2 is taken up in 1.5 mL of sterile water, 1.0 mL of a 1.0 M sodium phosphate solution obtained according to Example 12 and 2.5 mL of a 0.6 M sodium hydrogen carbonate solution obtained according to Example 23. The concentration of BMP-2 in the solution is 0.2 mg/mL. The solution is incubated for two hours at 4° C. The solution obtained is clear, and is filtered aseptically on a 0.22 μm membrane.

### EXAMPLE 27

### Preparation of a Solution of rhBMP-2 in the Presence of Sodium Phosphate and Sodium Hydrogen Carbonate

Solution 15: 88.4 mg of an rhBMP-2 lyophilizate in INFUSE buffer containing 3.7 mg of rhBMP-2 are taken up in 18.5 ml of a solution containing 0.23 M sodium phosphate and 0.31 M sodium hydrogen carbonate. The concentration of BMP-2 in the solution is 0.2 mg/mL. The solution is incubated for two hours at 4° C. The solution obtained is clear, and is filtered aseptically on a 0.22 μm membrane.

Examples of preparation of solutions comprising a soluble calcium salt and a base are given as non-limiting guides.

### EXAMPLE 28

### Solution of Calcium Chloride and Histidine

Solution 16: A solution containing 0.75 M calcium chloride and 0.4 M histidine is prepared by adding 112.5 mL of a 2 M calcium chloride solution, 120 mL of a 1 M histidine solution and 67.5 mL of deionized water. This solution is incubated for 30 minutes at room temperature and filtered aseptically on a 0.22 μm membrane.

### EXAMPLE 29

### Solution of Calcium Chloride and Proline

Solution 17: A solution containing 0.75 M calcium chloride and 0.75 M proline is prepared by adding 112.5 mL of a 2 M calcium chloride solution, 112.5 mL of a 2 M proline solution and 75 mL of deionized water. This solution is incubated for 30 minutes at room temperature and filtered aseptically on a 0.22 μm membrane.

### EXAMPLE 30

### Solution of Calcium Chloride and Glycine

Solution 18: A solution containing 0.75 M calcium chloride and 0.75 M glycine is prepared by adding 112.5 mL of a 2 M calcium chloride solution, 112.5 mL of a 2 M glycine solution and 75 mL of deionized water. This solution is incubated for 30 minutes at room temperature and filtered aseptically on a 0.22 μm membrane.

### EXAMPLE 31

### Solution of Calcium Chloride and Alanine

Solution 19: A solution containing 0.75 M calcium chloride and 0.75 M alanine is prepared by adding 112.5 mL of a 2 M calcium chloride solution, 112.5 mL of a 2 M alanine solution and 75 mL of deionized water. This solution is incubated for 30 minutes at room temperature and filtered aseptically on a 0.22 μm membrane.

### EXAMPLE 32

### Solution of calcium chloride and lysine

Solution 20: A solution containing 0.75 M calcium chloride and 0.75 M lysine is prepared by adding 112.5 mL of 2 M calcium chloride solution, 112.5 mL of a 2 M lysine solution and 75 mL of deionized water. This solution is incubated for 30 minutes at room temperature and filtered aseptically on a 0.22 μm membrane.

### EXAMPLE 33

### Solution of Calcium Chloride and Serine

Solution 21: A solution containing 0.75 M calcium chloride and 0.75 M serine is prepared by adding 112.5 mL of a 2 M calcium chloride solution, 112.5 mL of a 2 M serine solution and 75 mL of deionized water. This solution is incubated for 30 minutes at room temperature and filtered aseptically on a 0.22 μm membrane.

Examples of preparation of implants comprising a BMP, a soluble calcium salt, a soluble phosphate salt and/or a base are given as non-limiting guides.

The implants described in the following examples are prepared with a collagen sponge of sterile crosslinked type I such as Helistat (Integra LifeSciences, Plainsboro, N.J.). The volume of this sponge is 200 μL for an application to an ectopic site in rats.

### EXAMPLE 34

### Preparation of Collagen Sponge/rhBMP-2 Implants in the Presence of Lyophilized Calcium Chloride and Sodium Phosphate

Implant 1: 40 μl of a 0.05 mg/mL solution of BMP-2 obtained by diluting 28 μL of a 1.46 mg/mL solution in a 10 mM HCl buffer in 788 μL of sterile water are introduced into a sterile 200 mm3 crosslinked collagen sponge. The solution is incubated for 15 minutes in the collagen sponge, followed by adding 10 μl of a calcium chloride solution at a concentration of 1.64 M and 90 μL of a 0.47 M sodium phosphate solution. The sponge is then frozen and lyophilized aseptically. The dose of rhBMP-2 is 2 μg.

### EXAMPLE 35

### Preparation of Collagen Sponge/rhGDF-5 Implants in the Presence of Lyophilized Calcium Chloride, Sodium Phosphate and Histidine

Implant 2: 70 μl of a solution containing GDF-5 at 0.14 mg/mL and sodium phosphate at 0.11 M obtained by diluting 0.16 mL of a 4.0 mg/mL GDF-5 solution in 10 mM HCl buffer with 0.495 mL of sodium phosphate Solution 2 and 3.845 mL of sterile water. The solution is incubated for 15 minutes in the collagen sponge, followed by addition of 35 μl of a 0.17 mol/L histidine solution and finally 35 μL of a 0.38 M calcium chloride solution. The sponge is then frozen and lyophilized aseptically. The dose of rhGDF-5 is 10 μg.

### EXAMPLE 36

### Preparation of Collagen Sponge/rhBMP-2 Implants in the Presence of Lyophilized Calcium Chloride and Sodium Phosphate

Implant 3: 800 μl of Solution 15 are applied to a crosslinked type-I collagen sponge 4.5 cm3 in volume. The solution is incubated for 15 minutes in the collagen sponge, followed by addition of 800 μl of a 0.38 M calcium chloride solution. After the impregnation time, the sponge is ready for implantation. The dose of rhBMP-2 is 160 μg.

### Counterexample 1

### Preparation of a Collagen Sponge Implant Containing 20 μg of rhBMP-2

Implant 4: 40 μl of a 0.5 mg/ml solution of rhBMP-2 in a 10 mM HCl buffer are introduced aseptically into a sterile 200 mm3 crosslinked collagen sponge. The solution is left for 30 minutes in the collagen sponge before implanting.

The dose of rhBMP-2 in implant 2 is 20 μg.

### Counterexample 2

### Preparation of a Collagen Sponge Implant Containing 2 μg of rhBMP-2

Implant 5: 40 μl of a 0.05 mg/ml solution of rhBMP-2 in a 10 mM HCl buffer are introduced aseptically into a sterile 200 mm3 crosslinked collagen sponge of Helistat type (Integra LifeSciences, Plainsboro, N.J.). The solution is left for 30 minutes in the collagen sponge before implanting.

The dose of rhBMP-2 in implant 3 is 2 μg.

### Counterexample 3

### Preparation of Collagen Sponge Implants Containing 2.3 mg of rhBMP-2

Implant 6: Osteogenic implants were obtained by impregnating a crosslinked type-I collagen sponge 5.02×2.54×0.35 cm in size, i.e. a sponge volume of 4.52 mL, with 1600 μL of a 1.45 mg/mL solution of rhBMP-2, i.e. 2.3 mg. The solution is left for 30 minutes in the collagen sponge before implantation.

### Counterexample 4

### Preparation of Collagen Sponge Implants Containing 1.3 mg of rhBMP-2

Implant 7: Osteogenic implants were obtained by impregnating a crosslinked type-I collagen sponge 5.02×2.54×0.35 cm in size, i.e. a sponge volume of 4.52 mL, with 1600 μL of a 0.80 mg/mL solution of rhBMP-2, i.e. 1.3 mg. The solution is left for 30 minutes in the collagen sponge before implantation.

### EXAMPLE 37

### Evaluation of the Osteo-Inductive Power of the Various Formulations

The object of this study is to demonstrate the osteo-inductive power of the various formulations in a model of ectopic bone formation in rats. Male rats weighing 150 to 250 g (Sprague Dawley OFA-SD, Charles River Laboratories France, B.P. 109, 69592 l'Arbresle) are used for this study.

An analgesic treatment (buprenorphine, Temgesic®, Pfizer, France) is administered before the surgical intervention. The rats are anesthetized by inhalation of a mixture of O2 and isoflurane (1-4%). The fur is removed by shaving over a wide dorsal area. The skin of this dorsal area is disinfected with a povidone iodine solution (Vetedine® solution, Vetoquinol, France).

Paravertebral incisions of about 1 cm are made so as to expose the right and left paravertebral dorsal muscles. Access to the muscles is made by transfacial incision. Each of the implants is placed in a pocket such that no compression can be exerted thereon. Four implants are implanted per rat (two implants per site). The implant opening is then sutured using polypropylene yarn (Prolene 4/0, Ethicon, France). The skin is closed up using a non-absorbable suture. The rats are then returned to their respective cages and kept under observation during their recovery.

At 21 days, the animals are anesthetized by injection of tiletamine-zolazepam (ZOLETIL®25-50 mg/kg, IM, VIRBAC, France).

The animals are then sacrificed by injection of a dose of pentobarbital (DOLETHAL®, VETOQUINOL, France). Each site is then observed macroscopically, any sign of local intolerance (inflammation, necrosis, hemorrhage) and the presence of bony and/or cartilaginous tissue is recorded and rated according to the following scale: 0: absence, 1: weak, 2: moderate, 3: marked, 4: sizable.

Each of the explants is removed from its site of implantation and macroscopic photographs are taken. The size and weight of the explants are then determined. Each explant is then stored in buffered 10% formaldehyde solution.

Results:

This in vivo experiment makes it possible to measure the osteo-inducing effect of rhBMP-2 placed in a dorsal muscle of a rat. This non-bony site is said to be ectopic. The results of the various examples are collated in the following table.

| | Presence of bony tissue | Mass of explants (mg) |
|---|---|---|
| Implant 1 | 3.4 | 52 |
| Implant 4 | 3.6 | 38 |
| Implant 5 | — | — |

A dose of 20 μg of rhBMP-2 in a collagen sponge (Counterexample 1) leads to the production of ossified explants with an average mass of 38 mg after 21 days.

A dose of 2 μg of rhBMP-2 in a collagen sponge (Counterexample 2) does not have any osteo-inductive power that is sufficient for the collagen implants to be able to be found after 21 days.

When the rhBMP-2 is coprecipitated in the presence of calcium phosphate, a dose of rhBMP-2 of 2 μg (Example 28) makes it possible to generate ossicles, in contrast with rhBMP-2 alone at the same dose. Furthermore, these ossicles have a mass and a bone score equivalent to those with rhBMP-2 alone at a dose of 20 μg. This formulation thus makes it possible to greatly improve the osteogenic activity of rhBMP-2 with an equivalent effect at a 10 times lower dose.

### EXAMPLE 38

### Evaluation of the Osteoinductive Power of the Various Formulations in Posterolateral Fusion

The object of this study is to demonstrate the osteoinductive power of the various formulations in a model of posterolateral fusion in rabbits. This study is conducted according to the experimental protocol described in the publication by J P Lawrence (Lawrence, J. P. et al., Spine 2007, 32 (11), 1206-1213.) with the exception of the treatment with nicotine, since induction of a pseudoarthrosis is not desired.

The fusion of the vertebrae is evaluated by manual palpation of the explanted spinal column. The absence of mobility between the vertebrae is synonymous with fusion. The spinal column is also analyzed by micro-CT at 12 weeks to evaluate the presence of bone in the vertebrae. The results obtained for the various implants are summarized in the following table.

| | Protein | Dose of protein (mg) | Fusion |
|---|---|---|---|
| Implant 6 | BMP-2 | 2.3 | 2/2 |
| Implant 7 | BMP-2 | 1.3 | 7/8 |
| Implant 3 | BMP-2 | 0.16 | 4/4 |

From these posterolateral fusion studies in rabbits, it emerges that BMP-2 coprecipitated with the salt calcium phosphate makes it possible to reduce the doses of BMP-2 by a factor of 8 relative to the effective dose of BMP-2 in INFUSE buffer of 1.3 mg of BMP-2 for equivalent fusion results. Even at BMP-2 doses of 0.16 mg, posterolateral fusion is observed in all the rabbits in the case of BMP-2 coprecipitated with the salt calcium phosphate.

1. A coprecipitate consisting of at least one osteogenic protein in its insolubilized form and at least one insoluble calcium salt, said coprecipitate being in divided form.

2. The coprecipitate as claimed in claim 1, in which the insoluble calcium salt is chosen from the group consisting of calcium orthophosphates in anhydrous or hydrated form, alone or as a mixture.

3. The coprecipitate as claimed in claim 1, which also comprises at least one insoluble calcium salt chosen from the group consisting of calcium oxalate, calcium ascorbate, calcium carbonate and calcium sulfate.

4. The coprecipitate as claimed in claim 1, in which the insoluble calcium salt is chosen from the group consisting of mixed salts formed between cationic calcium ions and anionic ions such as mono-, di- or tribasic phosphates, polysaccharide carboxylates, carbonates, hydroxides and the possible anions borne by bases.

5. The coprecipitate as claimed in claim 1, which also comprises at least one growth factor with chemo-attracting and angiogenic power.

6. The coprecipitate as claimed in claim 1, in which the osteogenic protein is chosen from the group consisting of BMP-2 (dibotermine-alpha), BMP-4, BMP 7 (eptotermine-alpha), BMP-14 and GDF 5, alone or in combination.

7. The coprecipitate as claimed in claim 5, in which the at least one growth factor with chemo-attracting and angiogenic power is PDGF.

8. The coprecipitate as claimed in claim 5, which comprises at least BMP-2 and PDGF-BB.

9. The coprecipitate as claimed in claim 5, which comprises at least BMP-7 and PDGF-BB.

10. The coprecipitate as claimed in claim 5, which comprises at least GDF-5 and PDGF-BB.

11. The coprecipitate as claimed in claim 5, in which the osteogenic protein is chosen from the group consisting of BMP-2 (dibotermine-alpha), BMP-4, BMP 7 (eptotermine-alpha), BMP-14 and GDF-5, alone or in combination, and the at least one growth factor with chemo-attracting and angiogenic power is VEGF.

12. A kit for preparing an osteogenic implant, comprising at least:
a—a composition comprising at least one osteogenic protein,
b—a composition comprising at least one soluble calcium salt,
c—a composition comprising at least one soluble salt of an anion capable of forming an insoluble calcium salt.

13. The kit as claimed in the preceding claim, also comprising an additional composition comprising at least one base.

14. The kit as claimed in the preceding claim, also comprising a second base that may be added to compositions b, c or d.

15. The kit as claimed in claim 12, in which the composition comprising the osteogenic protein may also comprise the soluble salt of an anion capable of forming an insoluble calcium salt and/or a base.

16. The kit as claimed in claim 12, in which the composition comprising the soluble calcium salt may also comprise a base.

17. A kit comprising:
a—a composition comprising at least one osteogenic protein,
b—a composition comprising at least one base and at least one soluble salt of an anion capable of forming an insoluble calcium salt,
c—a composition comprising at least one soluble calcium salt.

18. A kit comprising:
a—a composition comprising at least one osteogenic protein,
b—a composition comprising at least one soluble salt of an anion capable of forming an insoluble calcium salt,
c—a composition comprising at least one soluble calcium salt and at least one base.

19. The kit as claimed in claim 12, in which the osteogenic protein is chosen from the group consisting of BMP-2 (dibotermine-alpha), BMP-4, BMP 7 (eptotermine-alpha), BMP-14 and GDF-5, alone or in combination.

20. The kit as claimed in claim 12, in which the composition comprising at least one osteogenic protein comprises at least one growth factor with chemo-attracting and angiogenic power.

21. The kit as claimed in claim 20, in which the composition comprising at least one osteogenic protein comprises at least one growth factor with chemo-attracting and angiogenic power.

22. The kit as claimed in claim 21, in which the growth factor with chemo-attracting and angiogenic power is PDGF.

23. The kit as claimed in claim 12, which comprises at least BMP-2 and PDGF-BB.

24. The kit as claimed in claim 12, which comprises at least BMP-7 and PDGF-BB.

25. Kit as claimed in claim 12, which comprises at least GDF-5 and PDGF-BB.

26. The kit as claimed in claim 12, in which the osteogenic protein is chosen from the group consisting of BMP-2 (dibotermine-alpha), BMP-4, BMP 7 (eptotermine-alpha), BMP-14 and GDF-5, alone or in combination, and the at least one growth factor with chemo-attracting and angiogenic power is VEGF.

27. The kit as claimed in claim 12, in which the soluble calcium salt is chosen from the group consisting of calcium chloride, D gluconate, formate, D-saccharate, acetate, L-lactate, glutamate and aspartate.

28. The kit as claimed in claim 12, in which the soluble calcium salt is calcium chloride.

29. The kit as claimed in claim 12, in which the soluble salt of an anion capable of forming a precipitate with the calcium ion is a soluble salt whose anion is chosen from the group consisting of phosphate anions comprising the phosphate ion PO43-, the hydrogen phosphate ion HPO42- and the dihydrogen phosphate ion H2PO4-.

30. The kit as claimed in claim 12, in which the base is chosen from mineral and organic bases.

31. The kit as claimed in claim 30, in which the mineral base is chosen from the group consisting of sodium hydroxide, sodium hydrogen carbonate and sodium carbonate.

32. The kit as claimed in claim 30, in which the organic base is chosen from the group consisting of amines and deprotonated amino acids.

33. The kit as claimed in claim 30, in which the organic base is chosen from the group consisting of imidazole and derivatives thereof, especially histidine, proline, ethanolamine and serine.

34. The kit as claimed in claim 12, which also comprises at least one organic matrix or a mineral matrix or a mixed matrix.

35. The kit as claimed in claim 34, in which the matrix is an organic matrix chosen from the group consisting of hydrogels and/or matrices based on a crosslinked polymer.

36. The kit as claimed in claim 35, in which the hydrogel is a hydrogel obtained by chemical or physical crosslinking of polymer chains.

37. The kit as claimed in claim 35, in which the crosslinked polymer is crosslinked and sterilized purified natural collagen.

38. The kit as claimed in claim 36, in which the hydrogel is chosen from the group of synthetic polymers including copolymers of ethylene glycol and of lactic acid, copolymers of ethylene glycol and of glycolic acid, poly(N vinylpyrrolidone), polyvinylic acids, and polyacrylamides and polyacrylic acids.

39. The kit as claimed in claim 35, in which the hydrogel is chosen from the group of natural polymers including hyaluronic acid, keratan, pullulan, pectin, dextran, cellulose and cellulose derivatives, alginic acid, xanthan, carrageenan, chitosan, chondroitin, collagen, gelatin, polylysine, fibrin, and biologically acceptable salts thereof.

40. The kit as claimed in claim 12, in which the compositions constituting the kit are aqueous solutions.

41. The kit as claimed in claim 12, in which the compositions constituting the kit are lyophilizates.

42. A process for preparing the coprecipitate as defined in claim 1, which comprises a coprecipitation step obtained by:
precipitating the osteogenic protein by addition of the solution of calcium ion salt,
precipitating the calcium ions by addition of a composition comprising at least one soluble salt of an anion capable of forming an insoluble calcium salt at a given pH,
the anionic polymer/osteogenic protein complex being obtained by adding the anionic polysaccharide solution to the osteogenic protein solution.

43. The process as claimed in claim 42, in which the precipitation of the calcium salt takes place in the form of calcium phosphate, by addition of a soluble phosphate solution.
